(19) United States
(12) Patent Application Publication
(10) Pub. No.: US 2014/0100290 A1
(43) Pub. Date: Apr. 10, 2014

(54) Method of treating a prostate condition

(71) Applicant: Jerome Rosenstock, (US)

(72) Inventor: Supriya A. Bavadekar, Woodside, NY (US)

(21) Appl. No.: 13/815,512

(22) Filed: Mar. 8, 2013

Related U.S. Application Data
(60) Provisional application No. 61/744,791, filed on Oct. 4, 2012.

(51) Int. Cl. A61K 31/05

(52) U.S. Cl. CPC A61K 31/05
USPC 514/731; 568/780

(57) ABSTRACT

A method of treating a prostate cancer condition in a mammal in need of such treatment is disclosed. The method comprises administrating to the mammal an anti-prostate cancer effective amount of a compound of a plant essential oil.

### This is a continuation-in-part of Provisional Patent Application No. 61/744,791 filed on Oct. 4, 2012

### BACKGROUND OF THE INVENTION

1. Field of the Invention

This invention relates to a method of treating a cancer condition of prostate cancer and more particularly, to a method of treating the prostate cancer condition in a mammal in need thereof by administrating to the mammal an anti-cancer effective amount of compound of a plant essential oil.

2. Discussion of the Prior Art

A cancer condition is a horrible disease which affects mammals, e.g. humans, worldwide. The form of treating a human patient varies according to the type of form of the cancer and how far advanced the cancer condition has progressed. In this regard it is to be noted that there are many types or forms of the cancer condition (estimated at about 200 types). It is also to be pointed out and stressed that effective treatment for one distinct type by a compound may be unsuccessful or ineffective for a different distinct type with the same compound.

Cancer is the leading cause of death in economically developed countries and the second leading cause of death in developing countries. One in four deaths in the United States is due to cancer. Prostate cancer is the most common noncutaneous malignancy affecting men in the United States.

Clinically, prostate cancer is diagnosed as local or advanced. Localized prostate cancer is the most commonly diagnosed stage. Prostate cancer starts as an androgen-dependent disease which requires presence of androgens to proliferate. Androgen ablation therapy is used for the treatment of patients with metastatic prostate cancer. However, following androgen ablation therapy, some cancer cells become androgen-independent and are able to grow in the low androgen environment. This is termed as hormone-refractory or androgen-independent prostate cancer. Current treatment options for localized, locally advanced, metastatic and hormone-refractory prostate cancer include chemotherapy, radiation therapy, hormonal therapy and surgery. There are, however, significant limitations associated with these, such as gastrointestinal toxic effects, genitourinary toxic effects, and urinary incontinence with radiotherapy.

The specific plant essential oils of this invention comprise a monocyclic, carbocyclic ring structure having six-members and substituted by at least one oxygenated or hydroxyl functional moiety. Examples of plant essential oils encompassed within the present invention, include, but are not limited to, members selected from the group consisting of aldehyde C16 (pure), amyl cinnamic aldehyde, amyl salicylate, anisic aldehyde, benzyl alcohol, benzyl acetate, cinnamaldehyde, cinnamic alcohol, a-terpineol, carvacrol, carveol, citral, citronellal, citronellol, p-cymene, diethyl phthalate, dimethyl salicylate, dipropylene glycol, eucalyptol (cineole), eugenol, iso-eugenol, galaxolide, geraniol, guaiacol, ionone, d-limonene, menthol, methyl anthranilate, methyl ionone, methyl salicylate, a-phellandrene, pennyroyal oil, perillaldehyde, 1- or 2-phenyl ethyl alcohol, 1- or 2-phenyl ethyl propionate, piperonal, piperonyl acetate, piperonyl alcohol, D-pulegone, terpinen-4-ol, terpinyl acetate, 4-tert butylcyclohexyl acetate, thyme oil, thymol, metabolites or trans-anethole, canillin, ethyl vanillin, and the like.

Carvacrol, a monoterpene phenol is the major constituent of the essential oil of oregano. Oregano is a collective term that includes members of several genera, including Thymus Origanum, Thymbra, and Satureia all containing carvacrol as the main constituent in their essential oils.

The anti-proliferative activities of this compound have been shown in a number of cancer cell lines including human metastatic breast cancer (MDA-MB-231 and MCF-7). However, its effect on prostate cancer has heretofore not been known, much less its effect on the human prostate carcinoma cells, LNCaP and PC-3.

The compounds of the plant essential oils are very effective on these cells lines in the treatment of a prostate cancer condition, affecting both the apoptosis and proliferative activities of these cell lines. This is especially true of the compound carvacrol.

### SUMMARY OF THE INVENTION

This invention relates to a method of treating a prostate cancer condition and more particularly, to a method of treating the prostate cancer condition in a mammal, e.g. a human, in need thereof by administrating to the mammal an anti-cancer effective amount of a compound of the plant essential oils.

### DETAILED DESCRIPTION

U.S. Pat. No. 7,291,650 B2 is incorporated by reference herein in its entirety.

Compounds of the plant essential oils have been found to cause inhibition of growth on cells LNCaP and PC-3, prostate cancer cells by inducing apoptosis in these cells. Thus the compounds of this invention are useful in treating a patient in need thereof by administering to the patient an anti-prostate cancer effective amount of the compound, its pharmaceutically acceptable salt thereof or any mixture of the foregoing.

As plant essential oil compounds are known and used for other purposes, they may be prepared by a skilled artisan by employing known methods. In addition, they may be purchased from conventional sources, may be readily isolated from specific plants or trees and purified (isolated) or may be synthesized using conventional techniques. Advantageously, these compounds may be conveniently synthesized from readily available starting materials. The relative ease with which the compositions of the present invention can be synthesized represents an enormous advantage in the large-scale production of these compounds.

It will be appreciated that the therapeutically-active plant essential oil compounds of the present invention may be modified or derivatized by appending appropriate functionalities, i.e. functional groups, to enhance selective biological properties. Such modifications are known in the art and include those that increase biological penetration into a given biological compartment (e.g. blood, lymphatic system, central nervous system), increase oral availability, increase solubility to allow administration by injection, alter metabolism and alter rate of excretion. In addition, the plant essential oil compounds may be altered to pro-drug form such that the desired therapeutically-active form of the compound is created in the body of the patient as the result of the action of metabolic or other biochemical processes on the pro-drug. Some examples of pro-drug forms include ketal, acetal, oxime, and hydrazone forms of compounds which contain ketone or aldehyde groups.

Moreover, the therapeutically-effective plant essential oil compounds of the present invention may contain one or more asymmetric carbon atoms and thus may occur as racemates and racemic mixtures, single enantiomers, diastereomeric mixtures and individual diastereomers. Each stereogenic carbon may be of the R or S configuration. All such isomeric forms of these compounds are expressly included within the purview of the present invention.

As will be appreciated, the compositions and method of the present invention include pharmaceutical compositions that comprise at least one plant essential oil, and pharmaceutically acceptable salts thereof, in combination with any pharmaceutically acceptable carrier, adjuvant or vehicle. The term “pharmaceutically acceptable carrier or adjuvant” refers to a carrier or adjuvant that may be administered to a patient, together with a plant essential oil compound of the present invention, and which does not destroy the pharmacological activity thereof and is nontoxic when administered in doses sufficient to deliver a therapeutic amount of the compound.

Pharmaceutically acceptable salts of the plant essential oil compounds of this invention include those derived from pharmaceutically acceptable inorganic and organic acids and bases. Examples of suitable acid salts include, without limitations, acetate, adipate, alginate, aspartate, benzoate, benzenesulfonate, bisulfate, butyrate, citrate, camphorate, camphorsulfonate, cyclopentanpropionate, digluconate, dodecylsufate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g. sodium), alkaline earth metal (eg. magnesium), ammonium and N-(C1-4 alkyl)4+ salts. The present invention also envisions the quaternization of any basic nitrogen-containing groups of the compounds disclosed herein. Water or oil-soluble or dispersible products may be obtained by such quaternization.

Further, pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d.alpha-toccopherol polyethyleneglycol 1000 systems, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, plyethylene glycol, sodium carboxymethylcellulose, polyarylates, waxes, polyethylene-polyoxyproplene-block polymers, polyethylene glycol and wool fat. Cyclodextrin, or chemically modified derivatives such as hydroxyalkylcyclodextrins, including 2- and 3-hydroxypropyl-beta-cyclodextrins, or other solublized derivatives may also be advantageously used to enhance delivery of therapeutically-effective plant essential oil compounds of the present invention.

The pharmaceutical compositions of this invention may be administered orally, parenterally, by inhalation spray, topically, rectally, nasally, bucally, vaginally or via an implanted reservoir, however, oral administration or administration by injection is preferred. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutanous, intravenous, intramuscular, intraarticular, intrasynovial, intrasternal, intrathecal, intralesional and intracranial injection or infusion techniques.

The pharmaceutical compositions may be in the form of sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluents or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and it glyceride derivatives are useful in the preparation of injectables, as are natural pharmaceutically—acceptable oils, such as olive oil or castor oil, especially in their polyoxyethylated versions. These oil solutions or suspensions may also contain a long-chain alcohol diluents or dispersant such as Ph. Helv or a similar alcohol.

The pharmaceutical compositions of the present invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration on a capsule form, useful diluents included lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of the present invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of the present invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention included, but are not limited to, mineral oil, liquid petroleum, white petroleum, propylene glycol, polyoxyethylene polyoxyproplene compound, emulsifying wax and water. Alternatively, the pharmaceutical composition can be formulated with a suitable lotion or cream containing the active compound suspended or dissolved in a carrier. Suitable carriers included, but are not limited to, mineral oil, sorbitan monostearate, polysorbate 60, cetyl esters wax, cetearyl alcohol, 2-octyidodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical composition of this invention may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Another acceptable pharmaceutical preparation would be an encapsulated form of the plant essential oils, as is, or modified as per the prior description. The walls of the capsules could be designed to release the plant essential oils rapidly, i.e. one minute, hour or day, or it could be designed to release over some designated period of time, i.e. days, weeks or months. The wall materials could be natural or synthetic polymers acceptable to the US FDA or composed of lipids or other suitable materials. These capsules could be delivered either orally or by injection and could be either water or oil based depending upon the desired method of use or required rate of release.

Dosage levels of between about 0.001 and about 100 mg/kg body weight per day, preferably between about 0.5 and about 75 mg/kg body weight per day of the active ingredient compound, for example, carvacrol are useful in the prevention and treatment of prostate cancer conditions. Typically, the pharmaceutical compositions of this invention will be administered from about 1 to 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain about 20% to about 80% active compound, e.g carvacrol.

Upon improvement of a patient's condition, a maintenance dose of a compound, composition or combination of this invention may be administered, if necessary. Subsequently, the dosage or frequency of administration, or both, may be reduced as a function of the symptoms, to a level at which the improved condition is retained when the symptoms have been alleviated to the desired level, treatment should cease. Patients may, however, require intermittent treatment on a long-term basis upon any recurrence of disease symptoms.

The prophylactic use of the present invention may require the daily intake of a prophylactically-effective amount.

As the skilled artisan will appreciate, lower or higher doses than those recited above may be required. Specific dosage and treatment regiments for any particular patient will depend upon a variety of factors, including the activity of the specific compound employed, the age, body weight, general health status, sex, diet, time of administration, rate of excretion, drug combination, the severity and course of a cancer, the patient's disposition to cancer and the judgment of the treating physician.

The following examples are for illustrative purposes and are not to be construed as limiting the invention disclosed herein. All temperatures are given in degrees centigrade.

In view of the amendments to the Manual of Patent Examining Procedure, including Sections 608.01(p); 707.07; 2004; 2012 dated January, 1981, Examples I to 13 of the specification are to be read as if they were expressed in the past tense since they are examples which have actually been carried out.

The inhibition of the growth of LNCaP prostate cancer cells by inducing apoptosis were determined using assays or tests described hereafter using the following materials.

### 1. Materials

Carvacrol (98% pure), 2-{[2-hydroxy-1,1-bis(hydroxymethyl)ethyl]amino}ethanesulfonic acid (TES), phosphate-buffered saline (PBS), ethyl alcohol (EtOH) (95%), penicillin-streptomycin solution, fetal bovine serum (FBS), Ponceau S solution, bovine serum albumin (BSA), protease inhibitor cocktail, isopropanol and boric acid were obtained from Sigma-Aldrich corp. (St. Louis, Mo., USA). PSVue™ 480 reagent kit was purchased from Molecular Targeting Technologies Inc. (WestChester, Pa., USA). XTT cell proliferation assay kit and RPMI-1640 medium were obtained from American Type Culture Collection (ATCC) (Manassas, Va., USA). Pierce®BCA protein assay kit, Pierce® ECL (enhanced chemiluminescence) western blotting substrate, ethidium bromide, Tris and EDTA were obtained from Fisher Scientific LLC (Hanover park, Ill., USA). Apoptotic DNA-Ladder Kit was obtained from Roche Applied Science (Mannheim, Germany). Trackit™ A-DNA/Hind III fragments were purchased from Life Technologies Inc. (Grand Island, N.Y., USA). Antibodies against PARP, cytochrome c, β-action and secondary horseradish peroxidase-conjugated antibody were purchased from Santa Cruz Biotechnology, Inc. (Santa Cruz, Calif., USA). TEMED, bis-acrylamide, 2-mercaptoethanol and nitrocellulose membrane were obtained from Bio-Rad laboratories (Hercules, Calif., USA). ProSieve QuadColor™ protein marker was obtained from Lonza Inc. (Me., USA). All other chemicals were purchased from Fisher Scientific LLC (Hanover park, Ill., USA), unless otherwise specified. Stock solutions (3×10⁻¹ M) of carvacrol were prepared fresh in ethanol and diluted in a mixture of water and ethanol on the day of experiments. The concentration of ethanol was maintained at 1.15% in all carvacrol test solutions. 1.15% ethanol was used as solvent control in all experiments.

The cell line and culture conditions were as follows:

Cell line and culture conditions:

Human metastatic prostate cancer cells, LNCaP, were obtained from ATCC (Manassas, Va., USA). Cells were grown in RPMI-1640 medium supplemented with 2mM glutamine, 10% FBS, 100 U/mL penicillin, and 100 μg/mL streptomycin. LNCaP cells were maintained in a humidified atmosphere of 5% CO2 at 37° C. in T-75 flasks and were subcultured when confluent. Experiments were conducted using cells with passage numbers ranging from 5-20.

### EXAMPLE 1

### Cell Viability Assay

The anti-proliferative effect of carvacrol was assessed in human metastatic prostate cancer cells, LNCaP, using the XTT cell proliferation assay. This assay is a colorimetric assay that detects cellular metabolic activities. In this assay, the second generation tetrazolium dye XTT (2,3-bis (2-methoxy-4-nitiro-5-sulfophenyl)-5-[(phenylamino)-carbonyl]-2H tetrazolium hydroxide), is reduced to a soluble, brightly orange colored formazan derivative. To improve the sensitivity of XTT assay, PMS (N-methyl dibenzopyrazine methyl sulfate), an intermediate electron carrier is used. This color change is accomplished by breaking apart the positively charged quaternary tetrazole ring. Mitochondrial oxidoreductases present in viable cells are thought to contribute substantially to the XTT response with their reductants being transferred to the plasma membrane.

LNCaP cells at the density of 10,000 cells/well were seeded into flat-bottom, 96-well microtiter plates (100 μL/well) and allowed to attach and grow for 24 hours, the cells were then treated with 0.1 μM, 1 μM, 10 μM, 100 μM, 1000 μM, and 3000 μM of carvacrol for 24, 48, and 96 hours. In the 96 hour treatment protocol, medium and drug were replaced after 48 hours. Activated XTT solution was added to each well (50 μL/well) of the plates after completion of the treatment period, and allowed to incubate further for 4 hours at 37° C. After incubation, an orange colored formazan product was formed and the absorbance was read at 475 nm using BioTek microplate reader. Viability in solvent control (1.15% EtOH)-treated cells was considered to be 100%. Cell viability of the drug-treated cells was calculated as a fraction of solvent control and the anti-proliferative activity of carvacrol was expressed in terms in IC₅₀ valuesIC₅₀ values of carvacrol were calculated using GraphPad Prism 4 (GraphPad Software Inc., Calif., USA), and are expressed as the means of ±S.E.M> of n≧4 experiments, with triplicate determinations in each experiment. One-way analysis of variance (ANOVA) was used to analyze the data followed by Tukey's post hoc test. Differences were considered statistically significant if p<0.05.

Results from cell viability studies showed that LNCaPcell proliferation was inhibited after treatment with 10⁻⁷ M-3×10⁻³ M of carvacrol at all three incubation periods tested, viz. 24, 48, and 96 hours. Carvacrol inhibited the growth of LNCaP cells in both, a time- and dose-dependent manner. IC₅₀ of carvacrol was found to be 1668.93±178.75 μM, 268.97±58.01 μM and 13.72±4.42 μM following 24, 48 and 96 hours treatment, respectively.

### EXAMPLE 2

### Detection of Phosphatidylserine Externalization Using Fluorescence Microscopy

One of the earliest events to occur in apoptosis or programmed cell death is the externalization of phosphatidylserine (PS), a membrane phospholipid restricted to the inner leaflet of the lipid bilayer. Redistribution of PS from the inner leaflet to the outer leaflet occurs in cells undergoing apoptosis. The PSVue™ 480 reagent kit was used to perform the phosphatidylserine (PS) externalization assay. The principle behind this assay involves a strong binding of PSVue™ 480 (fluorescent probe) to the anionic PS residues exposed on the surface of the apoptotic cells by its Zn (II)-dipicolylamine (Zn-DPA) functionality, making it a useful apoptotic sensor.

To detect PS externalization, LNCaP cells were seeded as 0.5×10⁶ cells/well in flat-bottom, 6-well microtiter plates. After 24 hours of incubation at 37° C., cells were treated with 10 μM and 1000 μM of carvacrol for 48 hours. At the end of the treatment period, cells were washed twice with TES buffer (5 mM TES, 145 mM NaCl, pH=7.4), labeled with 10 μM PSVue™ 480 dye, and allowed to incubate for 30 seconds. After incubation, cells were again washed twice with TES buffer and externalization of PS was observed using a fluorescent microscope equipped with FITC filter set-Axiovert 200M (Carl Zeiss MicroImaging Inc, NY, USA). 1.15% EtOH was used as a solvent control.

LNCaP cells treated with 10 μM and 1000 μM of carvacrol for 48 hours showed externalization of phosphatidylserine residues that was detected using the PSVue™ 480 dye. Increase in the amount of green fluorescence (PSVue™ 480 positive cells) following conjugation of PSVue™ 480 labeled cells with FITC, in a dose-dependent manner, suggested the induction of apoptosis by carvacrol.

### EXAMPLE 3

### Morphological Analysis of Cells

Prominent features of morphological changes occurring in a cell, such as cell shrinkage, rounding of cells, membrane blebbing, and formation of apoptotic bodies indicate induction of apoptosis or programmed cell death.

In order to identify the morphological changes in LNCaP cells after treatment with carvacrol, 2×10⁶ cells/well were seeded in flat-bottom, 6-well microtiter plates, allowed to grow for 24 hours, treated with 10 μM and 1000 μM of carvacrol, and incubated for a further 48 hours at 37° C. Post incubation, cells were washed twice with PBS, and microscopic photographs of morphological changes of the cells were captured using a fluorescent microscope, Axiovert 200M (Carl Zeiss MicroImaging Inc, NY, USA). 1.15% EtOH was used in solvent control.

Data showed that typical features of apoptosis like rounding of cells, membrane blebbing, cell shrinkage and formation of apoptotic bodies were observed in carvacrol-treated cells in a dose-dependent manner.

### EXAMPLE 4

### DNA Fragmentation Assay

During apoptosis, pro-apoptotic proteins such as apoptosis-inducing factor (AIF), endonuclease G, and caspase-activated DNase (CAD) are released from the mitochondria, which occur after the cell has committed to die. AIF, after translocation to the nucleus, causes DNA fragmentation (˜50-300 kb pieces) and peripheral nuclear chromatin condensation which is referred to as “stage 1” condensation. Endonuclease G also cleaves nuclear chromatin to produce oligonucleosomal DNA fragments following its translocation to the nucleus. Both, AIF and endonuclease G function in caspase-independent manner. CAD subsequently released from mitochondria, after getting cleaved by activated caspase-3, is known to translocate into the nucleus leading to oligonucleosomal cleavage of DNA into 180 by fragments and a more pronounced chromatin condensation referred to as “stage 11” condensation.

This cleaved DNA is easily observed as a “ladder” upon analysis by gel electrophoresis. This DNA fragmentation is considered to be a biochemical hallmark of apoptosis.

In this study, apoptotic DNA-Ladder Kit was used to detect DNA fragmentation in LNCaP cells. The cells were plated as 2×10⁶ cells/wall in flat-bottom, 6-well microtiter plates, allowed to grow for 24 hours, and then treated with 10 μM and 1000 μM of carvacrol for 48 hours. Cells were harvested after 48 hours and DNA was isolated using the Apoptotic DNA-Ladder Kit. DNA preparations were electrophoresed in 1% agarose gels, stained with ethidium bromide (0.5 μg/mL) and visualized under UV light. Trackit™ A-DNA/Hind 111 fragments (0.1 μg/mL) were used as a standard, and the image was captured using a Polaroid camera.

Fragmentation of DNA was observed in LNCaP cells treated with 10 μM and 1000 μM of carvacrol for 48 hours, indicating apoptosis.

### EXAMPLE 5

### Western Blot Analyses to Determine the Expression of Cytochrome c Release and PARP Cleavage

Western blotting is a widely used analytical technique for detection of specific proteins separated from one another, according to their size, by gel electrophoresis. Application of an electric current induces proteins on the gel to move on to the membrane (nitrocellulose or PVDF) placed next to it. Proteins on the gel get transferred on to the membrane which is subsequently stained with the antibody. Once detected, the target protein will be visualized as a band on a blotting membrane, X-ray film, or an imaging system.

Cytochrome c is a heme-containing component of the electron transport chain in the mitochondrial membrane. It is bound to the outer surface of the inner mitochondrial membrane. During apoptosis, cytochrome c is released from the mitochondria into the cytosol in response to pro-apoptotic stimuli such as Bax protein.

Poly-ADP ribosylation of a variety of nuclear proteins is catalyzed by PARP with NAD as substrate. PARP gets activated upon DNA damage. PARP, a 116 kDa nuclear protein which normally functions in DNA damage detection and repair, is cleaved by caspase 3 and caspase 7 between Asp214 and Glyn215 to yield 89 kDa and 24 kDa fragments.

For Western blot analyses, LNCaP cells were plated as 2×10⁶ cells/well in flat bottom, 6-well microtiter plates and allowed to grow for 24 h. They were then treated with 10 μM and 1000 μ4 of carvacrol for 48 h in order to determine the release of cytochrome c and cleavage of PARP. Cells were harvested after 48 h, washed twice with ice-cold PBS and lysed with lysis buffer containing 20 mM Tris pH 8.0, 1 mM EDTA, 150 mM NaCl, 1% NP-40, 1 mM (3-glycerophosphate, 0.5% sodium deoxycholate, 1 mM sodium orthovanadate and phosphatase inhibitors. Protease inhibitor cocktail was also added to the lysis buffer in a 1:100 dilution.

After maintaining constant agitation at 4° C. for 30 minutes, lysates were collected in microcentrifuge tubes and centrifuged at 12,000 rpm at 4° C. for 20 minutes. Supernatants were used as whole cell extracts. Protein concentration was determined using the Pierce® BCA protein assay kit. The proteins (6 μg) were separated on 8-15% SDS—polyacrylamide gels along with protein molecular weight standards. They were then transferred to a nitrocellulose membrane. Transfer was checked using 10% Ponceau S solution in deionized water. The membranes were blocked by 5% BSA solution and then probed with relevant antibody (PARP and cytochrome c at a 1:500 dilution) for 12-16 h at 4° C. with constant agitation. After incubation with the primary antibody, the membranes were washed three times with Tris-Buffered Saline Tween-20 (TBST) and then probed with horseradish peroxidase-conjugated secondary antibody (1:10000) at room temperature for 1 h with constant shaking. The membranes were incubated for 1 minute in ECL Western blotting substrate after washing them again with TBST three times, and the image was captured using the Gel Logic 2200 PRO system (Carestream Molecular Imaging, Conn., USA). β-actin antibody (1:5000) was used as a loading control. 1.15% EtOH was used as a solvent control. Densitometric analyses were performed using the Carestream Molecular Imaging software. Values expressed were compared to solvent control which was taken as 100%. Western blot images are representative of at least 3 independent experiments, while quantification data represent mean±S.E.M. of n>3 experiments.

Western blot analyses showed a dose-dependent increase in cytochrome c release in LNCaP cells after treatment with 10 μM and 1000 μM of carvacrol for 48 h. Percent relative increase in cytochrome c release was more in cells treated with carvacrol compared to cells treated with solvent control (1.15% EtOH), as determined by densitometric analysis. Results of Western blot analyses also demonstrated cleavage of PARP in carvacrol-treated LNCaP cells following 48 h treatment.

The inhibition of growth of PC-3 cells by inducing apoptosis were determined using assays or tests described hereafter using the following materials.

Materials
Carvacrol (98% pure), 2-{[2-hydroxy-1,1-bis(hydroxymethymethyl]amino}ethanesulfonic acid (TES), phosphate-buffered saline (PBS), ethyl alcohol (EtOH) (95%), penicillin-streptomycin solution, fetal bovine serum (FBS), Ponceau S solution, bovine serum albumin (BSA), protease inhibitor cocktail, isopropanol and boric acid were obtained from Sigma-Aldrich corp. (St. Louis, Mo., USA). PSVue™ 480 reagent kit was purchased from Molecular Targeting Technologies Inc. (WestChester, Pa., USA). XTT cell proliferation assay kit was obtained from American Type Culture Collection (ATCC) (Manassas, Va., USA). Pierce® BCA protein assay kit, Pierce® ECL (enhanced chemiluminescence) western blotting substrate, ethidium bromide, Tris and EDTA were obtained from Fisher Scientific LLC (Hanover park, Ill., USA). Apoptotic DNA Ladder Kit was obtained from Roche Applied Science (Mannheim, Germany). Antibodies against cytochrome c, β-actin and secondary horseradish peroxidase conjugated antibody were purchased from Santa Cruz Biotechnology, Inc. (Santa Cruz, Calif., USA). TEMED, bis-acrylamide, 2-mercaptoethanol and nitrocellulose membrane were obtained from Bio-Rad laboratories (Hercules, Calif., USA). ProSieve QuadColor™ protein marker was obtained from Lonza Inc. (Me., USA). All other chemicals were purchased from Fisher Scientific LLC (Hanover park, Ill., USA), unless otherwise specified. Stock solutions (3×10⁻¹ M) of carvacrol were prepared fresh in ethanol and diluted in a mixture of water and ethanol on the day of the experiments. The concentration of ethanol was maintained at 1.15% in all carvacrol test solutions. 1.15% ethanol was used as solvent control in all experiments.

Cell Line and Culture Conditions
Human metastatic, androgen insensitive prostate cancer cells, PC-3, were obtained from ATCC (Manassas, Va., USA). Cells were grown in F-12K medium supplemented with 2 mM glutamine, 10% FBS, 100 U/mL penicillin, and 100 μg/mL streptomycin. PC-3 cells were maintained in a humidified atmosphere of 5% CO₂ at 37° C. in T-75 flasks and were subcultured when confluent.

### EXAMPLE 6

### Cell Viability Assay

The anti-proliferative effect of carvacrol was assessed in PC-3 cells using the XTT cell proliferation assay. This assay is a colorimetric assay that is used to measure cell growth and drug sensitivity in tumor cell lines by measuring the internal environment of the proliferating cell, which is more reduced than that of non-proliferating cells. In this assay, the second generation tetrazolium dye, XTT (2,3-bis(2-methoxy-4-nitro-5-sulfophenyl)-5-[(phenylamino)-carbonyl]-2H tetrazolium hydroxide), is reduced to a soluble, brightly orange colored formazan derivative. To improve the sensitivity of XTT assay, PMS (N-methyl dibenzopyrazine methyl sulfate), an intermediate electron carrier is used. This color change is accomplished by breaking apart the positively charged quaternary tetrazole ring. An expansion in the number of viable cells results in an increase in the overall activity of mitochondrial dehydrogenases in the sample. This augmentation in the enzyme activity leads to an increase in the amount of formazan dye formed, which directly correlates to the number of metabolically active cells in the culture.

PC-3 cells at a density of 10⁴ cells/well were seeded into flat-bottom, 96-well microtiter plates (100 μL/well) and allowed to attach and grow for 24 h. After 24 h, the cells were treated with 10 μM, 100 μM, 300 μM, 1000 μM, 2000 μM and 3000 μM of carvacrol for 24, 48, and 96 h. In the 96 h treatment protocol, medium and drug were replaced with fresh medium and drug solutions after 48 h. Activated XTT solution was added to each well (50 μL/well) of the plates after completion of the treatment period, and allowed to incubate further for 4 hours at 37° C. After incubation, an orange colored formazan product was formed and the absorbance was measured at 475 nm using BioTek microplate reader. Viability in solvent control (1.15% EtOH)-treated cells was considered to be 100%. Cell viability of the drug-treated cells was calculated as a fraction of solvent control and the anti-proliferative activity of carvacrol was expressed in terms of IC₅₀ values. IC₅₀ values were calculated using GraphPad Prism 4 (Graph Pad Software Inc., Calif., USA), and expressed as the means±S.E.M. of n≧3 experiments (each in triplicate).

The anti-proliferative effect of carvacrol on PC-3 cells determined using XTT cell proliferation assay revealed a dose-dependent and time-dependent decrease in cell growth following treatment with carvacrol. The IC₅₀ value for carvacrol was determined to be 2250.53±79.78 μM, 1395.93±98.17 μM and 1007.7±63.51 μM following 24, 48, and 96 hr treatment, respectively.

### EXAMPLE 7

### Detection of Phosphatidylserine Externalization Using Fluorescence Microscopy

One of the earliest events to occur in apoptosis or programmed cell death is externalization of phosphatidylserine (PS), a membrane phospholipid restricted to the inner leaflet of the lipid bilayer. Redistribution of PS from inner leaflet of plasma membrane lipid bilayer to the outer leaflet occurs in cells undergoing apoptosis. The PSVue™ 480 reagent kit was used to perform the phosphatidylserine (PS) externalization assay. The principle behind this assay involves a strong binding of PSVue™ 480 (fluorescent probe) to the anionic PS residues exposed on the surface of apoptotic cells by its Zn (II)-dipicolylamine (Zn-DPA) functionality, making it a useful apoptotic sensor.

To detect PS externalization, PC-3 cells were seeded at a density of 0.5×10⁶ cells/well in flat-bottom, 6-well microtiter plates and were allowed to incubate for 24 h. After 24 h of incubation at 37° C., cells were treated with 10 μM and 1000 μM of carvacrol for 48 h. At the end of the treatment period, cells were washed twice with TES buffer (5 mM TES, 145 mM NaCl, pH=7.4), labeled with 10 μM PSVue™ 480 dye, and allowed to incubate for 30 seconds. After incubation, cells were again washed twice with TES buffer and externalization of PS was observed using a fluorescent microscope equipped with FITC filter set, Axiovert 200M (Carl Zeiss MicroImaging Inc, NY, USA). 1.15% EtOH was used as a solvent control.

PC-3 cells treated with 10 μM and 1000 μM of carvacrol for 48 hours showed externalization of phosphatidylserine residues that was detected using the PSVue™ 480 dye. A dose-dependent increase in the amount of green fluorescence (PSVue™ 480 positive cells) following treatment with carvacrol suggested the induction of apoptosis by carvacrol in these cells.

### EXAMPLE 8

### Morphological Analysis of Cells

During the early process of apoptosis, cells exhibit considerable morphological changes such as cell shrinkage, rounding of cells, membrane blebbing, and formation of apoptotic bodies. These typical features of apoptosis were used to evaluate the mechanism of decreased cell proliferation by carvacrol in PC-3 cells.

In order to identify the morphological changes in PC-3 cells after treatment with carvacrol, 2×10⁶ cells/well were seeded in flat-bottom, 6-well microtiter plates, allowed to grow for 24 h, then treated with 10 μM and 1000 μM of carvacrol, and incubated further for 48 h at 37° C. After incubation, cells were washed twice with PBS, and microscopic photographs of morphological changes of the cells were captured using a fluorescent microscope, Axiovert 200M (Carl Zeiss MicroImaging Inc, NY, USA). 1.15% EtOH was used as a solvent control.

PC-3 cells treated with 10 μM and 1000 μM of carvacrol for 48 h exhibited typical features of apoptosis like rounding of cells, membrane blebbing, and cell shrinkage.

### EXAMPLE 9

### Western Blot Analyses to Determine the Expression of Cytochrome c Release

Western blotting is a widely used analytical technique for detection of specific proteins separated from one another according to their size by gel electrophoresis. Application of an electric current induces negatively charged proteins on the gel to move on to the membrane (nitrocellulose or PVDF) placed next to it. Proteins on the gel get transferred on to the membrane which is subsequently probed with a primary antibody followed by detection using a secondary antibody, which are labeled with a probe to allow for detection. Once detected, the target protein will be visualized as a band on a blotting membrane, X-ray film, or an imaging system.

Cytochrome c is a heme-containing component of the electron transport chain in the mitochondrial membrane. It is bound to the outer surface of the inner mitochondrial membrane. During apoptosis, cytochrome c is released from mitochondria into the cytosol in response to the loss of membrane stability and pro-apoptotic stimuli such as Bax protein. In order to further confirm that carvacrol induced apoptosis in these cells, the release of cytochrome c from mitochondria into cytosol was analyzed using Western blot analysis in carvacrol-treated cells.

For Western blot analyses, PC-3 cells were plated at a density of 2×10⁶ cells/well in flat bottom, 6-well microtiter plates and allowed to adhere for 24 h at 37° C. They were then treated with 10 μM and 2000 μM of carvacrol for 48 h in order to determine the release of cytochrome c. Cells were harvested after 48 h, washed twice with ice cold PBS and lysed with lysis buffer containing 20 mM Tris pH 8.0, 1 mM EDTA, 150 mM NaCl, 1% NP-40, 1 mM β-glycerophosphate, 0.5% sodium deoxycholate, 1 mM sodium orthovanadate and phosphatase inhibitors. Protease inhibitor cocktail was also added to the lysis buffer at a 1:100 dilution.

After maintaining constant agitation at 4° C. for 30 minutes, lysates were collected in microcentrifuge tubes and centrifuged at 12,000 rpm at 4° C. for 20 minutes. Supernatants were used as whole cell extracts. Protein concentration was determined using the Pierce® BCA protein assay kit. The proteins (6 μg) were separated on 8-15% SDS—polyacrylamide gels along with protein molecular weight standards. They were then transferred to a nitrocellulose membrane. Transfer was checked using 10% Ponceau S solution in deionized water. The membranes were blocked by 5% BSA solution and then probed with a primary antibody for cytochrome c at 1:500 dilution for 12-16 h at 4° C. with constant agitation. After incubation with primary antibody, the membranes were washed three times with TBS and then probed with horseradish peroxidase-conjugated secondary antibody at room temperature for 1 hour with constant shaking. The membranes were incubated for 1 minute in ECL western blotting substrate and the image was captured using Gel Logic 2200 PRO (Carestream Molecular Imaging, CT, USA). β-actin antibody was used as a loading control to check for equal amount of protein loading in each lane. 1.15% EtOH was used as a solvent control. Densitometric analyses were performed using the Carestream Molecular Imaging software. Values expressed were compared to solvent control which was taken as 100%. Western blot images are representative of at least 3 independent experiments, while quantification data represent mean±S.E.M. of n≧3 experiments.

Our data showed that carvacrol induced release of cytochrome c in PC-3 cells following treatment with carvacrol. Increase in cytochrome c levels in cytosol was observed in cells treated with 10 μM carvacrol, as compared to solvent control, indicating induction of apoptosis. There was a decrease in levels of cytochrome c observed with higher concentration (2000 μM) of carvacrol as compared to 10 μM carvacrol-treated cells.

I claim:

1. A method of treating a prostate cancer condition in a mammal in need thereof, which comprises,
administering to the mammal an effective amount of a plant essential oil compound.

2. The method as defined in claim, where said a mammal is a human being.

3. The method as defined in claim 2 wherein said compound is carvacrol.

4. A method to affect the apoptosis of prostate cancer cells comprising LNCaP cells, PC-3 cells and any mixture of these cells which compromises,
treating said cells with a compound selected from the group consisting of carvacrol, a pharmaceutically acceptable salt of carvacrol or a mixture thereof in an apoptosis effective amount.

5. An anti-prostate cancer composition which compromises an anti-prostate cancer effective amount of a monoterpene phenol selected from carvacrol, a pharmaceutically acceptable salt of carvacrol or a mixture of the foregoing.

6. A method to affect apoptosis or cell proliferation of a prostate cancer cell selected from the group consisting of LNCaP, PC-3, or any mixture of the foregoing, which comprises,
treating the cell with a plant essential oil compound in apoptosis or cell anti-proliferation effective amount.

7. An apoptosis or anti-proliferation effective composition for prostate cancer cells selected from the group consisting of LNCaP, PC-3, or any mixture of the foregoing which comprises a compound selected from the group consisting of carvacrol, a pharmaceutically acceptable salt thereof or any mixture of the foregoing.

8. A method of preventing a prostate cancer condition which comprises,
administering to a mammal an effective amount of a plant essential oil compound.
